(19) United States
(12) Patent Application Publication
(10) Pub. No.: US 2026/0060802 A1
(43) Pub. Date: Mar. 5, 2026

(54) AORTIC VALVE REPLACEMENT PROSTHESIS

(71) Applicant: NANJING SAINT MEDICAL TECHNOLOGY CO., LTD., Nanjing (CN)

(72) Inventor: Chenming MA, Nanjing (CN)

(21) Appl. No.: 19/380,326

(22) Filed: Nov. 5, 2025

Related U.S. Application Data
(63) Continuation-in-part of application No. 17/758,270.

(30) Foreign Application Priority Data
Jan. 9, 2020 (CN) 202010021982.8

(51) Int. Cl. A61F 2/24; A61F 2/915

(52) U.S. Cl. CPC A61F 2/2418; A61F 2002/91583; A61F 2210/0076

(57) ABSTRACT

An aortic valve replacement prosthesis that is delivered through a catheter and does not depend only on friction for fixation. According to the device, multiple supporting arms (50) are provided on an intermediate portion (102) of a tubular body (105); the supporting arms are “D”-shaped after full expansion, and are fixed between a narrowest part (73) of the aorta close to the heart and a narrowest part (74) on an aortic annulus (70), so as to achieve sufficient match between the outer surfaces of the support arms (50) and surrounding tissues; each supporting arm (50) comprises three landing areas (54) and two bending sections (59). The present invention can accurately control the position of a valve to be released on the aortic annulus (70), and thus, adverse events caused by existing fixation relying solely on friction are avoided, thereby better curing aortic valve diseases.

### CROSS-REFERENCE TO RELATED APPLICATION

The present application is a continuation-in-part application of U.S. patent application Ser. No. 17/758,270, filed on Jun. 30, 2022, which is a U.S. national entry of PCT International Application No. PCT/CN2020/121635, filed on Oct. 16, 2020, which claims the priority of Chinese Patent No. 202010021982.8 filed on Jan. 9, 2020, with National Intellectual Property Administration, titled “STRUCTURALLY FITTED TRANSCATHETER AORTIC VALVE IMPLANTATION DEVICE”, the content of which is incorporated herein by reference in its entirety.

### TECHNICAL FIELD

The present disclosure relates to the field of medical devices, and in particular, to an aortic valve replacement prosthesis that is implantable via an approach through the aorta or through a transapical approach, which can be implanted via a catheter device.

### BACKGROUND

About 300 thousand people worldwide are affected by cardiac valve diseases each year. Such diseases involve abnormal leaflet tissues, e.g., excess tissue growth, tissue degeneration or rupture, tissue hardening or calcifying, or abnormal tissue position throughout the cardiac cycle (i.e., annular dilation or ventricular reshaping), leading to dysfunction of valve, e.g., leakage or blood backflow (i.e., valve insufficiency) or resistance to forward blood flow (i.e., valve stenosis).

At present, existing transcatheter aortic valves rely on the inherent properties of the stent material and are simply secured at the position of the original aortic valve by friction. For example, Patent No. CN107890382A discloses a locatable and retrievable transcatheter aortic valve, wherein a first funnel opening structure of a valve stent is in contact with the left ventricular outflow tract and aortic annulus to serve as a support, and the valve stent has a locating rod structure configured for axial locating by securing the valve stent via friction between the lower part of the valve stent and surrounding tissue. However, due to the complexity of the pathological structures, the valve stent is positioned and secured by friction only, which may cause the valve to be pulled and pressed by the original structure after implantation and may cause valve migration, resulting in the risks of embolization, falling off or ejection, thereby causing failure of the valve implantation operation.

### SUMMARY

The invention therefore intends to provide an aortic valve replacement prosthesis that is not solely fixed by friction. It forms a structural match with the blood vessels via a special design, accurately releases the valve at the aortic annulus, and avoids the adverse events caused by the existing fixation by friction alone, thus curing the aortic valve diseases. The invention is implemented by the following technical solutions:

The invention provides an aortic valve replacement prosthesis comprising a valve stent, a plurality of valve leaflets, an inner skirt and an outer skirt,
wherein the valve stent is radially compressible and re-expandable so as to be implanted via a catheter device, and the valve stent comprises a tubular body made of a metal grid and having a circumference extending along a longitudinal axis;
a first longitudinal end portion facing, in an implanted state, the ascending-aorta side of the native aortic valve;
a second longitudinal end portion facing, in an implanted state, the ventricular side of the native aortic valve; and
an intermediate portion disposed between the first and second longitudinal end portions;
wherein the tubular body has an inner circumferential surface defining an inner cavity of the tubular body and an outer circumferential surface defining an outer surface of the tubular body, the inner and outer circumferential surfaces extending substantially concentrically along the longitudinal axis;
wherein a plurality of support arms are provided on the intermediate portion of the tubular body, the support arms being spaced from each other around the circumferential direction of the tubular body, and the support arms being integrally formed with the tubular body, without being welded or fastened thereto;
the plurality of valve leaflets are disposed on the intermediate portion of the inner cavity of the tubular body;
the inner skirt is affixed to the second longitudinal end portion of the inner cavity of the tubular body and fixedly connected with the valve leaflets;
the outer skirt is affixed to the second longitudinal end portion of the outer surface of the tubular body and fixedly connected with the inner skirt;
and each of the support arms comprises a lower support arm, a first bending section, a platform section, a second bending section, and an upper support arm that are connected in sequence,
each of the lower support arm, the platform section, and the upper support arm comprises a lower, middle and upper landing area, respectively;
wherein each bending section is formed as a single, continuously contoured portion that, between two adjacent landing areas, has a narrow middle portion and smoothly flared end regions, the narrow middle portion having a width smaller than that of the landing areas, and the flared end regions merging gradually into the landing areas so as to avoid a sharp step and to facilitate bending of the support arm.

The term “substantially concentrically” refers to the inner and outer circumferential surfaces being coaxial with the longitudinal axis within manufacturing tolerance, such that the tubular body maintains an overall symmetrical structure, for example, with an angular misalignment of not more than about 2-3 degrees.

According to the aortic valve replacement prosthesis disclosed herein, each landing area has a substantially constant width along the circumferential direction, and the circumferential widths of the lower landing area, the middle landing area and the upper landing area are substantially equal.

The term “substantially constant width” means that the circumferential width of a landing area remains generally uniform along its length, with only minor variations attributable to manufacturing tolerances.

According to the aortic valve replacement prosthesis disclosed herein, the plurality of support arms are distributed equidistantly or non-equidistantly around the circumferential direction of the tubular body.

According to the aortic valve replacement prosthesis disclosed herein, the lower support arm and the upper support arm in each support arm each have a first end connected to the tubular body that is narrower in width than a second end connected to the corresponding lower or upper landing area of the support arm.

According to the aortic valve replacement prosthesis disclosed herein, the tubular body and the plurality of support arms are machined by laser cutting.

According to the aortic valve replacement prosthesis disclosed herein, in an expanded state, the lower support arm forms an angle of 45-55 degrees with respect to the outer surface of the tubular body, such that the support arms are appropriately oriented for anchoring at the aortic annulus and achieving optimal engagement with surrounding tissues. According to the aortic valve replacement prosthesis disclosed herein, the tubular body comprises a plurality of grid nodes, connected through a plurality of grid elements. The intermediate portion comprises a first node, a second node and a third node that are axially spaced apart along the tubular body. For each support arm, the lower support arm is affixed to the second node, and the upper support arm is affixed to the first node. The third node is located between the first node and the second node, and the grid elements between the first nodes and the second nodes to which the support arms are attached have a length greater than that of the grid elements between the first nodes and the second nodes to which the support arms are not attached.

According to the aortic valve replacement prosthesis disclosed herein, the inner skirt and the outer skirt are made of animal pericardium or other biocompatible materials having similar flexibility and sealing properties.

According to the aortic valve replacement prosthesis disclosed herein, a minimum width between the support arms and the adjacent grid elements allows passage of only one laser beam during laser cutting, thereby maximizing the landing area of the support arms.

It should be noted that the dimensions and/or sizes used herein for describing the valve stent generally refer to a free expanded state of the valve stent, i.e., the expanded state other than any compressed circumstance. Thus, the dimensions and/or locations in a re-expanded implanted state may be different due to the compression provided by surrounding tissues.

### Beneficial Effects of Present Disclosure

The present disclosure has advantages that structural matched of the aortic valve replacement prosthesis in the operation process is realized through the support arm structure located on the intermediate portion of the stent tubular body, thus reducing the risks of falling off, displacement or ejection in the process of implantation and increasing the success rate of valve implantation.

### BRIEF DESCRIPTION OF THE DRAWINGS

FIG. 1 shows a structural schematic view of an aortic valve replacement prosthesis according to the present disclosure;

FIG. 2A-2C are schematic views illustrating the positional relationship among the inner skirt, the outer skirt, and the valve leaflets of the aortic valve replacement prosthesis according to the present disclosure;

FIG. 3 shows a schematic view of an embodiment of the aortic valve replacement prosthesis according to the present disclosure;

FIG. 4 shows a detailed view of a support arm of the aortic valve replacement prosthesis according to the present disclosure;

FIG. 5 shows another detailed view of the support arm of the aortic valve replacement prosthesis according to the present disclosure;

FIG. 6 shows a schematic view of the overall function formed by a plurality of support arms of the aortic valve replacement prosthesis according to the present disclosure;

FIG. 7 shows a schematic view of a delivery device for the transcatheter aortic valve replacement prosthesis according to the present disclosure; and

FIG. 8 shows a schematic view of the two-dimensional machining process of the intermediate portion and the support arms of the aortic valve replacement prosthesis according to the present disclosure.

### DETAILED DESCRIPTION

The present disclosure will be further illustrated with reference to the following specific examples. It should be understood that these examples are merely intended to illustrate the present disclosure rather than limit the protection scope of the present disclosure. In addition, it should be understood that various changes or modifications may be made by those skilled in the art after reading the teachings of present disclosure, and these equivalents also fall within the protection scope of the present disclosure.

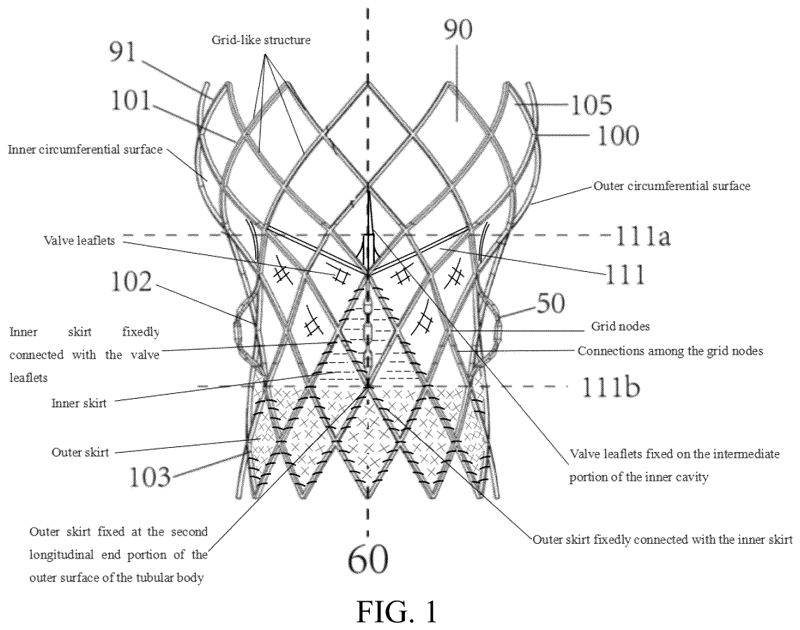

As shown in FIG. 1, a valve stent 100 comprises a tubular body 105, and the tubular body 105 consists of three portions, a first longitudinal end portion 101, an intermediate portion 102 and a second longitudinal end portion 103, which are made of a grid-like structure. The material of the tubular body 105 can be, for example, iron, nickel, aluminum, titanium, and/or alloys of these metals, and other elements. Reference numeral 111 denotes transparent artificial leaflet, each artificial leaflet 111 being connected to the inner cavity 90 of the tubular body 105.

For ease of description, two imaginary axial reference planes, 111a and 111b, are defined with respect to the longitudinal axis 60; they are not physical components of the device. A region between the two axial leaflets horizontal planes 111a and 111b that are longitudinally spaced apart from each other along the axis 60 of the tubular body 105 is a leaflet fixing region, wherein the axial leaflet horizontal plane 111a faces the first longitudinal end 101 and the axial leaflet horizontal plane 111b faces the second longitudinal end 103. The axial leaflet horizontal plane 111a spaces the first longitudinal end 101 apart from the intermediate portion 102. The axial leaflet horizontal plane 111b may be located around the second longitudinal end 103.

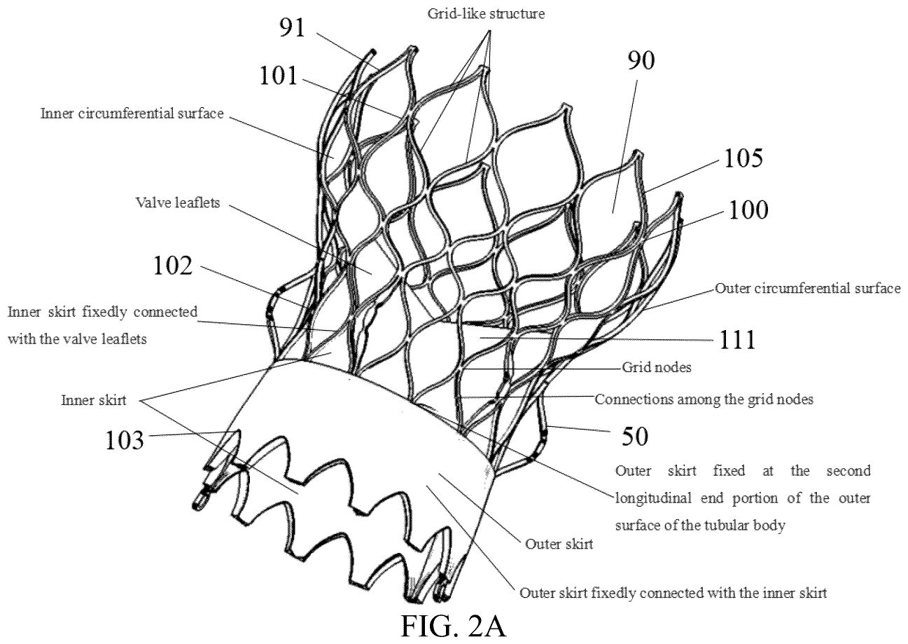

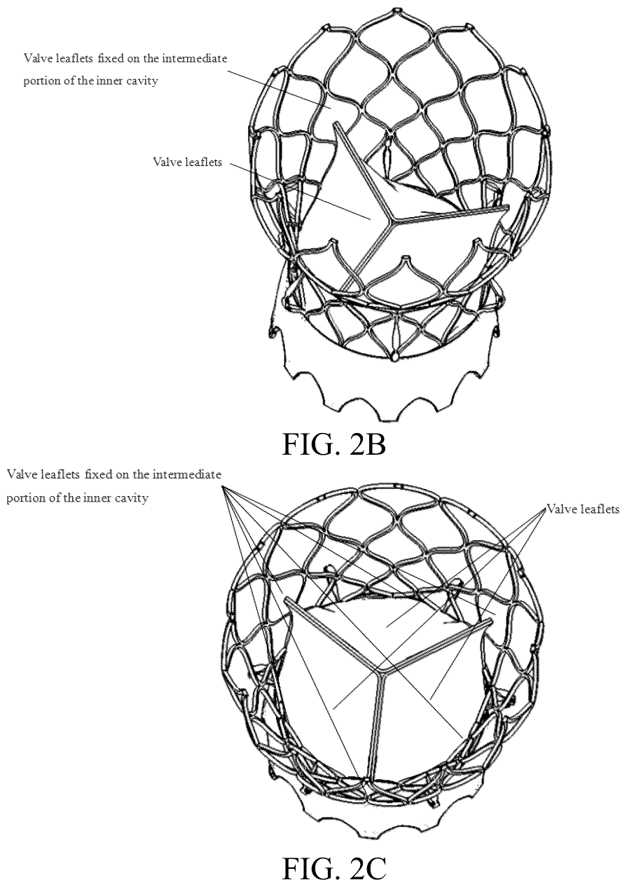

The valve stent 100 comprises an outer skirt and an inner skirt made of animal pericardium or artificial material. As shown in FIGS. 1, 2A, 2B and 2C, the inner skirt disposed along the inner circumferential surface of the tubular body 105 at the second longitudinal end portion 103, extending continuously around the circumference. Its upper edge is positioned immediately below the lower edges of the valve leaflets 111. The inner skirt is secured to the valve leaflets 111 and to the tubular body 105 by sutures, thereby providing a fluid-tight seal within the inner cavity 90. Its lower edge extends toward the distal rim of the second longitudinal end portion 103 and is circumferentially sutured to the lowermost grid cells of the tubular body 105 (e.g., along one or more contiguous rings of grid nodes), thereby anchoring the inner skirt and cooperating with the outer skirt to provide a two-sided sealing cuff.

The outer skirt is disposed along the outer circumferential surface of the same second longitudinal end portion 103, extending circumferentially in correspondence with the inner skirt. The outer skirt is sutured to the tubular body 105, and its lower edge extends slightly farther toward the distal rim of the stent to cover lower grid nodes and form an external sealing cuff on the outer surface 91.

The inner and outer skirts are joined by sutures passed through corresponding grid openings of the tubular body 105 so that the inner skirt is affixed to the inner cavity 90 at the second longitudinal end portion 103 and fixedly connected with the valve leaflets 111, and the outer skirt is affixed to the outer surface 91 at the same end portion 103 and fixedly connected with the inner skirt, thereby providing an annular sealing structure that enhances fixation stability and reduces paravalvular leakage.

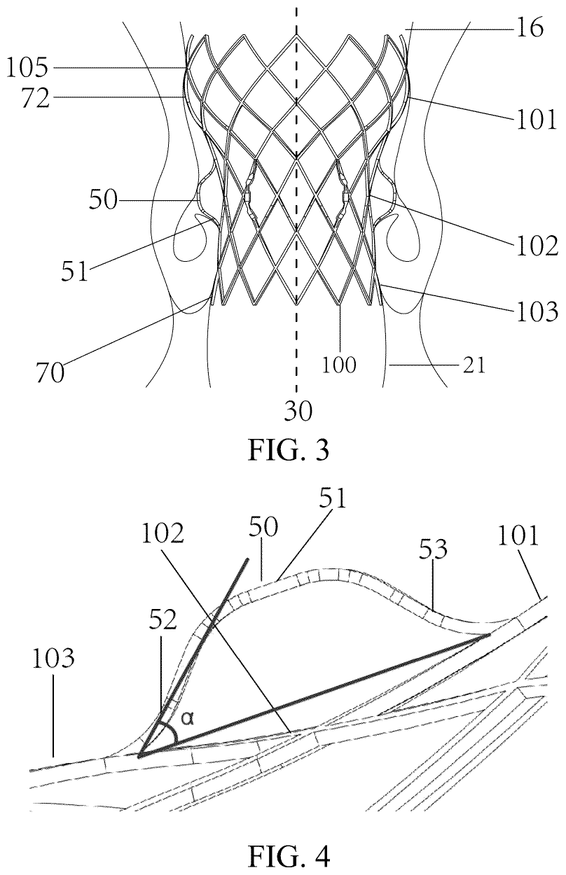

FIG. 3 shows an embodiment of the valve stent 100 for replacing a native aortic valve in a human or animal heart. That is, the valve stent 100 can be used as an artificial valve that allows blood to generally flow through the connecting channel in only one direction, in this embodiment, from the left ventricle 21 to the aorta 16, and may prevent leakage of blood in the direction from the aorta 16 to the left ventricle 21. The virtual longitudinal axis 30 is the longitudinal axis of the entire blood vessel. When the valve stent 100 is implanted, i.e., in its implanted state, the first longitudinal end portion 101 faces the aorta 16 ipsilaterally to the ascending aorta 72, and the second longitudinal end portion 103 faces the left ventricle 21 ipsilaterally to the native aortic annulus 70.

The implanted valve stent 100 is movable in its expanded state in the direction towards the aortic side 16, with the support arms 50 protruding toward the outer surface 91 of the tubular body 105. Thereby the support arms 50 move longitudinally over the native aortic annulus 70 under its radial compression. As the support arms 50 have a specific profile and are free of hooks, barbs, kinks, etc., the support arms 50 do not become entangled with the body's native tissues or cause tissue damage when moving longitudinally.

Referring to FIG. 4, the valve stent 100 comprises a plurality of support arms 50, such as 3, 6, 9, 12 or more. The support arms 50 are integrally formed rather than being joined together by welding or other synthetic process. When the valve stent 100 expands, the support arms 50 may be circumferentially spaced from one another. The support arms 50 may be spaced from one another by equal circumferential distances, i.e., equidistantly distributed around the tubular body 105, or the support arms 50 may be spaced from one another by unequal circumferential distances. Each support arm 50 has an overall “D” shape and consists of three portions, a platform section 51, an upper support arm 53 and a lower support arm 52. The “D” shaped design provides better integrity and anchoring function for structural matched in the transition region between the vessel and valve. The upper support arm 53, the lower support arm 52 and the platform section 51 are centrosymmetrical. The upper support arm 53 is connected to the intermediate portion 102 towards the first longitudinal end portion 101, and the lower support arm 52 is connected to the intermediate portion 102 towards the second longitudinal end portion 103, the upper and lower support arms being formed tangentially with a smooth transition. The platform section 51 is provided parallel to the direction of blood flow, rather than perpendicularly to the horizontal plane, and is capable of contacting the anatomical structure in the transition region of the vessel and valve in a parallel manner, minimizing the influence on blood flow.

As shown in FIG. 4, two angles are involved in the three-dimensional structure of the support arms, an upper angle and a lower angle. The upper angle and the lower angle are different in size. The lower angle α ranges from 45 degrees to 55 degrees, of which the selection is important for acquiring a D-shaped support arm with a maximum supporting force.

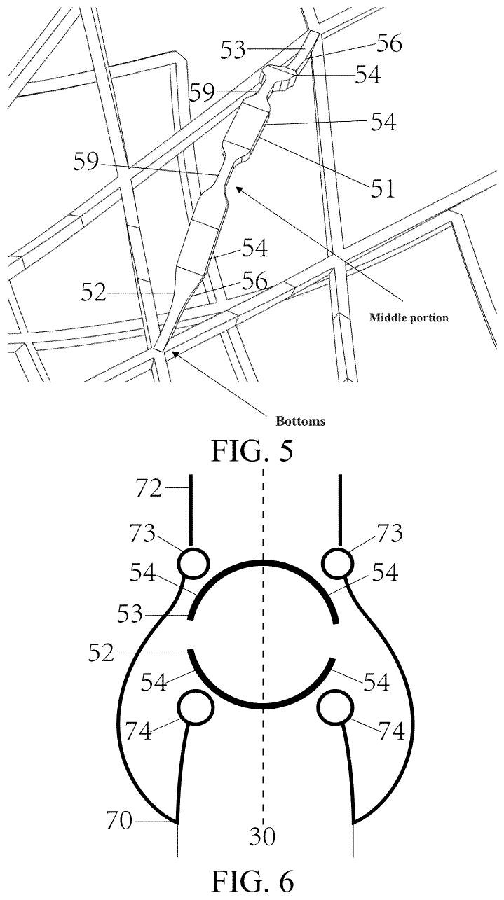

As shown in FIG. 5, the support arm 50 is further enlarged to better illustrate the formation of the structural match function. It can be seen that the upper support arm 53, lower support arm 52 and platform section are provided with landing areas 54 for acquiring greater tension and/or compression when match occurs in the heart or vessel. Connections 56 between the support arm 50 and the tubular body 105 are smoothly flared toward the tubular body so as to form a gradual transition from the landing areas 54 to the bottoms. Each bending section 59 is formed as a single, continuously contoured portion that, between two adjacent landing areas 54, includes a narrow-width region and smoothly flared end regions, such that the support arm 50 can be naturally bent to form a D-shaped configuration.

FIG. 6 schematically illustrates the function of the entirety formed by the plurality of support arms 50. The valve stent (not shown) forms an upper structural matched with the narrowest part 73 of the aorta close to the heart (e.g., the sinotubular junction, or other part of the ascending aorta) via the landing area 54 of the upper support arm 53. The valve stent (not shown) forms a lower structural matched with the narrowest part 74 above the aortic annulus (e.g., free edges of the aortic leaflet or the stenotic structures of the leaflet) via the landing area 54 of the lower support arm 52. This is clearly distinguished from existing prostheses implantation theories in the prior art relying solely on fixation by friction. The great risk of surgical failure due to fixation problems, such as displacement, falling off and ejection, is avoided by the structural matched.

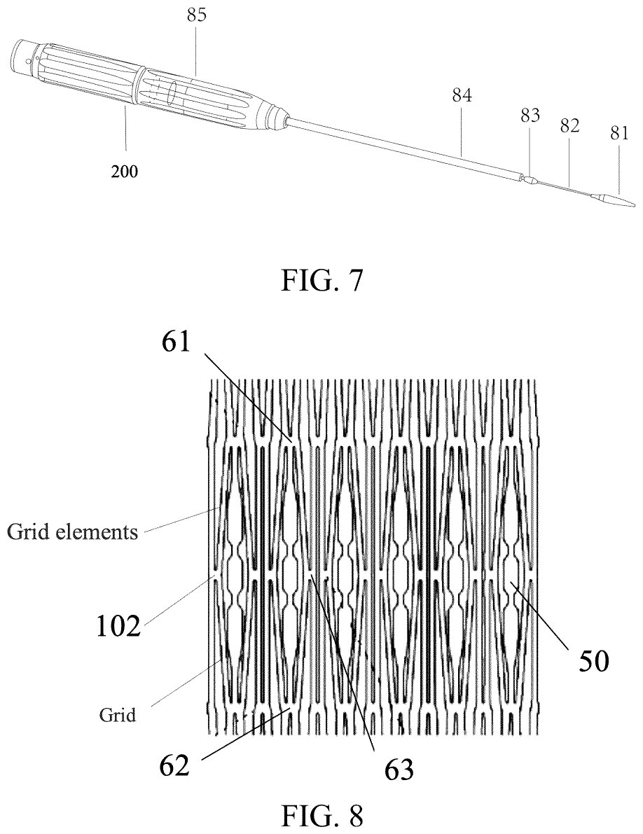

Referring to FIG. 7, a schematic view of a delivery device 200 for the transcatheter aortic valve replacement prosthesis of the present disclosure is shown. The delivery device 200 of the embodiment of the present disclosure comprises: a delivery head 81, a prosthesis loading area 82, a loading site 83, a delivery catheter 84 and a rotatable grip handle 85. By the mating of the loading site 83 and the delivery head 81, the valve stent 100 is received in the prosthesis loading area 82. By slowly rotating the rotatable grip handle 85, the delivery catheter 84 will be advanced slowly (or retracted) to advance (or retract) the rotatable grip handle to complete the receiving (or release) of the valve stent from the prosthesis loading area 82.

FIG. 8 is a schematic view of the two-dimensional machining process of the intermediate portion 102 and the support arms 50 of the tubular body, with the tubular body 105 and the support arms 50 being machined by laser cutting. The intermediate portion 102 of the tubular body 105 is provided with a plurality of grid nodes, connections among the grid nodes are grid elements, the grid nodes are divided into a first node 61, a second node 62 and a third node 63 according to axial positions, two ends of each of the support arms 50 are connected to the first node 61 and the second node 62 respectively, the third node 63 is located between the first node and the second node, and the grid elements between the first nodes and the second nodes to which the support arms are attached have a length greater than that of the grid elements between the first nodes and the second nodes to which the support arms are not attached. The process design allows an easier natural bending of the support arms 50 to a “D shaped configuration when the valve stent 100 expands.

Also, a minimum width between the support arms 50 and the adjacent grid elements allows passage of only one laser beam during laser cutting, thereby maximizing the landing area 54 of the support arms 50. A larger landing area 54 provides an increased contact surface with the anatomical structures in the transition region of the vessel and valve, enabling a desired distribution of tension and thereby facilitating the structural matching between the valve stent and the vessel wall.

Each landing area 54 has a substantially constant width along the circumferential direction, and the circumferential widths of the lower, middle and upper landing areas 54 are substantially equal, allowing the support arm 50 to bend naturally into the “D” shaped configuration when the valve stent 100 expands.

In the grid structure of the tubular body, different structural widths may be designed at different grid nodes to accommodate varying radial forces during expansion.

Examples of the present disclosure have been described above. However, the present disclosure is not limited to the above examples. Any modification, equivalent, improvement and the like made without departing from the spirit and principle of the present disclosure shall fall within the protection scope of the present disclosure.

1. An aortic valve replacement prosthesis comprising a valve stent (100), a plurality of valve leaflets, an inner skirt and an outer skirt,
wherein the valve stent (100) is radially compressible and comprises a tubular body (105) made of a metal grid and having a circumference extending along a longitudinal axis (60);
wherein the tubular body (105) comprises a first longitudinal end portion (101), a second longitudinal end portion (103), and an intermediate portion (102) disposed between the first and second longitudinal end portions (101, 103),
wherein the tubular body (105) has an inner circumferential surface defining an inner cavity (90) of the tubular body (105) and an outer circumferential surface defining an outer surface (91) of the tubular body (105), the inner and outer circumferential surfaces extending substantially concentrically along the longitudinal axis (60);
wherein a plurality of support arms (50) are provided on the intermediate portion (102) of the tubular body (105) and are spaced from each other around a circumferential direction of the tubular body (105), and are integrally formed with the tubular body (105), without being welded or fastened to the tubular body (105);
the plurality of valve leaflets are disposed on the intermediate portion (102) of the inner cavity (90) of the tubular body (105),
the inner skirt is affixed to the second longitudinal end portion (103) of the inner cavity (90) of the tubular body (105) and fixedly connected with the valve leaflets,
the outer skirt is affixed to the second longitudinal end portion (103) of the outer surface (91) of the tubular body (105) and fixedly connected with the inner skirt, and
each support arm (50) comprises a lower support arm (52), a first bending section (59), a platform section (51), a second bending section (59), and an upper support arm (53) that are connected in sequence,
each of the lower support arm (52), the platform section (51), and the upper support arm (53) comprises a lower landing area (54), a middle landing area (54), and an upper landing area (54), respectively,
wherein each bending section (59) is a single, continuously contoured portion having a narrow middle portion between two adjacent landing areas (54) and smoothly flared end regions that merge gradually into the landing areas (54) so as to avoid a sharp step and to facilitate bending of the support arm (50).

2. The aortic valve replacement prosthesis according to claim 1, wherein each landing area (54) has a substantially constant width along the circumferential direction, and the circumferential widths of the lower landing area (54), the middle landing area (54) and the upper landing area (54) are substantially equal.

3. The aortic valve replacement prosthesis according to claim 2, wherein the plurality of support arms (50) are distributed equidistantly or non-equidistantly around the circumferential direction of the tubular body (105).

4. The aortic valve replacement prosthesis according to claim 3, wherein the lower support arm (52) and the upper support arm (53) in each support arm (50) each have a first end connected to the tubular body (105) that is narrower in width than a second end connected to the corresponding lower or upper landing area (54) of the support arm (50).

5. The aortic valve replacement prosthesis according to claim 1, wherein the tubular body (105) and the plurality of support arms (50) are machined by laser cutting.

6. The aortic valve replacement prosthesis according to claim 1, wherein the metal grid comprises a plurality of grid nodes connected through a plurality of grid elements, and
wherein the intermediate portion (102) comprises a first node (61), a second node (62) and a third node (63) that are axially spaced apart along the tubular body (105),
and wherein the lower support arm (52) of each support arm (50) is affixed to the second node (62), and the upper support arm (53) of each support arm (50) is affixed to the first node (61).

7. The aortic valve replacement prosthesis according to claim 6, wherein, in an expanded state, the lower support arm (52) forms an angle of 45-55 degrees with respect to the outer surface (91) of the tubular body (105).

8. The aortic valve replacement prosthesis according to claim 1, wherein the inner skirt and the outer skirt are made of animal pericardium.
